(19) United States
(12) Patent Application Publication
(10) Pub. No.: US 2014/0305442 A1
(43) Pub. Date: Oct. 16, 2014

(54) FIXTURE FOR IMMOBILIZING AN ARM OF A PATIENT

(71) Applicant: ALL OF IT SCANDINAVIA AB, Bjärred (SE)

(72) Inventors: Hampus Bergenudd, Bjärred (SE); Per Ekdahl, Lund (SE)

(21) Appl. No.: 14/358,624

(22) PCT Filed: Nov. 16, 2012
(86) PCT No.: PCT/SE2012/000188

§ 371 Date: May 15, 2014

(30) Foreign Application Priority Data
Nov. 16, 2011 (SE) 1100856-2

(51) Int. Cl. A61B 19/00; A61M 25/01; A61B 6/12

(52) U.S. Cl. CPC A61B 19/203; A61B 6/12; A61M 25/0108
USPC 128/845

(57) ABSTRACT

A fixture for immobilizing an arm of a patient for insertion of a catheter for heart investigation and intervention. The fixture comprises a first portion (41) intended to be arranged at the upper arm (3), a second portion (42, 44, 45) intended to be arranged along the forearm and a third portion (43) intended to be arranged at the wrist with the wrist in an opened and twisted position. The second portion is divided into a first part (44) connected to the first portion (41) at an angle of about 150 degrees and a second part (45) connected to the third portion (43) at an angle of about 210 degrees. The first part (44) and the second part (45) are interconnected by a semi-bracelet (46) or a flexible strap (63). In this way, the first portion may be arranged at the inside of the elbow and the third portion may be arranged at the hand back at the opposite side of the arm. The fixture is attached to the arm via several flexible straps. The fixture is adapted to immobilize the arm and the wrist in a position adjacent the navel, in which the radial artery of the arm is conveniently available for catheterization.

### FIELD OF INVENTION

The present invention relates to a fixture for an arm of a patient. The fixture is especially intended to be used during X-ray investigation and evaluation and during possible balloon expansion of coronary vessels of the heart.

### BACKGROUND

Heart deceases are the most common cause of death in the Western World.

The most common heart decease is coronary artery obstruction. The coronary arteries provide oxygenated blood to the heart muscle itself. Obstructions result in less blood flow, often resulting in angina pectoris, which is chest pain due to ischemia, i.e. lack of blood supply, thus a lack of oxygen supply of the heart muscle, generally due to obstruction or spasm of the coronary arteries.

In order to confirm coronary vessel decease, the coronary vessels are exposed to X-ray investigation, which today is considered to be the “Golden Standard”. If obstructions or constrictions are found, such constrictions may be treated by balloon expansion or surgery, such as by-pass surgery, or by other methods. Obstructions, such as blood clots, may be dissolved by introduction of suitable agents.

The X-ray investigation and evaluation and other interventions may be performed by inserting a catheter extending to the heart via an artery, such as arteria femoralis or arteria radialis.

When the investigation takes place via the radial artery, it may be convenient for the surgeon to use the right arm of the patient lying in a supine position, since the surgeon almost always is placed at the right side of the patient. However, if the patient has been exposed to a previous by-pass surgery, an implant is often arranged in a vessel adjacent the clavicle. The right arm cannot be used for such patients but the left arm radial artery has to be used. Also of other reasons, it may be advantageous to use the left arm instead of the right arm of the patient.

When the left arm is used and the surgeon is arranged at the right side of the patient, the arm should be placed on the abdomen of the patient approximately at the middle of the patient. If the left arm is placed in this position, the palm of the hand is normally faced downward towards the abdomen and the radial artery at the wrist is not available. The arm needs to be rotated at least about 90 degrees around its symmetry axis and the wrist should be turned backward to expose the inner wrist portion, where the radial artery is most easily available. There is a need for a fixture for obtaining such a position.

However, if conventional fixtures for arms, and especially the elbow portion of the arm, is used, such fixtures normally comprises straps extending around the arm for attachment of the fixture to the arm. Such straps have a tendency to strangle the underlying artery and make difficult and even impossible to insert a catheter via the radial artery and further to the heart. Thus, there is a need for a new type of fixtures, that does not strangle the underlying artery.

### SUMMARY OF THE INVENTION

Accordingly, an object of the present invention is to mitigate, alleviate or eliminate one or more of the above-identified deficiencies and disadvantages singly or in any combination.

In an aspect, there is provided a fixture for being attached to an arm of a patient, comprising a first portion, a second portion and a third portion, the first portion being intended to be arranged adjacent an elbow at the inner side of the elbow and extending from the elbow up along an upper arm portion and being arranged integral with the second portion at a first angle; the second portion having a length which is smaller than or equal to a forearm and supporting the forearm; the third portion being integral with or connected to the second portion at a second angle.

In an embodiment, the second angle is in a direction opposite the first angle, and is about 190 degrees to 250 degrees and wherein the first and second angles are in substantially the same plane and wherein the fixture substantially forms a Z-shape.

In another embodiment, the second angle is in the same direction as the first angle, and is about 110 degrees to 170 degrees and wherein the first and second angles are in substantially the same plane and wherein the fixture forms a ground U-shape. The fixture may be attached to the arm of the patient by several flexible straps. The length of the second portion may be adjustable.

In a further embodiment, the second portion nay be divided in two parts interconnected by a semi-bracelet, whereby a first part of the second portion is arranged integral with said first portion by said first angle and a second part of the second portion is integral with or connected to the third portion at said second angle.

The semi-bracelet may extend over about 180 degrees around a symmetry axis of the arm, whereby the first part may be arranged at the inner side of the arm and the second part may be arranged at the outer side of the arm.

At least a portion of the fixture may be substantially transparent to X-ray radiation. At least a portion of member of the fixture may be sterilized.

In another further embodiment, the fixture may further comprise a fastening means for fastening the fixture in relation to the body of the patient, such as a fastening means adjacent the third portion intended for attachment to a belt.

In another aspect, there is provided a use of a fixture as mentioned above for immobilizing an arm of a patient at a position adjacent a navel of the patient and with a wrist accessible by having the wrist arranged in a twisted position with the thumb facing upwards or downwards when the patient is in a supine position, in order to facilitate insertion of a catheter in an artery of the arm for heart investigation or intervention. In addition, the wrist may be angled so that the wrist is opened up.

### BRIEF DESCRIPTION OF THE DRAWINGS

Further objects, features and advantages of the invention will become apparent from the following detailed description of embodiments of the invention with reference to the drawings, in which:

FIG. 1 is a schematic view of a patient lying in a supine position with his left hand in a resting position at his abdomen.

FIG. 2 is a schematic view similar to FIG. 2 in which the desired position of the hand and the wrist is shown.

FIG. 3 is a schematic view similar to FIG. 1 in which a first embodiment of a fixture is attached to the arm.

FIG. 4 is a side view of the first embodiment of the fixture.

FIG. 5 is a schematic view similar to FIG. 1 in which a first embodiment of a fixture is attached to the arm.

FIG. 6 is a side view of the first embodiment of the fixture.

FIG. 7 is a schematic view similar to FIG. 5 in which an adjustment strap is arranged.

FIG. 8 is a side view of a second embodiment of the fixture.

FIG. 9 is a schematic view similar to FIG. 1 of a third embodiment of the fixture.

FIG. 10 is a schematic view similar to FIG. 5 in which an adjustment belt is arranged.

FIG. 11 is a side view of the adjustment belt.

### DETAILED DESCRIPTION OF EMBODIMENTS

Below, several embodiments of the invention will be described. These embodiments are described in illustrating purpose in order to enable a skilled person to carry out the invention and to disclose the best mode. However, such embodiments do not limit the scope of the invention. Moreover, certain combinations of features are shown and discussed. However, other combinations of the different features are possible within the scope of the invention.

When a catheter should be used for X-ray investigation and possible balloon expansion, the most common way to insert such a catheter has up to now been insertion into the femoral artery, since it is sufficiently large and extends directly to the aortic arc and subsequently to the coronary arteries. However, since the femoral artery is large, there is also a concomitant risk of blood leakage and complications.

As an alternative, the use of the radial artery in the arm has been suggested. The surgeon is normally placed at the right side of the patient and has a heavy lead apron in order to shield the surgeon from X-rays. Thus, it may be convenient for the surgeon to use the right arm and difficult and cumbersome to use the left arm of the patient. However, the use of the left arm results in that the catheters may be arranged in a more convenient position. In addition, the use of the right arm may be impossible due to previous interventions. Thus, the embodiments shown below are intended to be used at the left arm of the patient when the surgeon is placed on the right side of the patient. However, the different embodiments may also be used at the right arm of the patient.

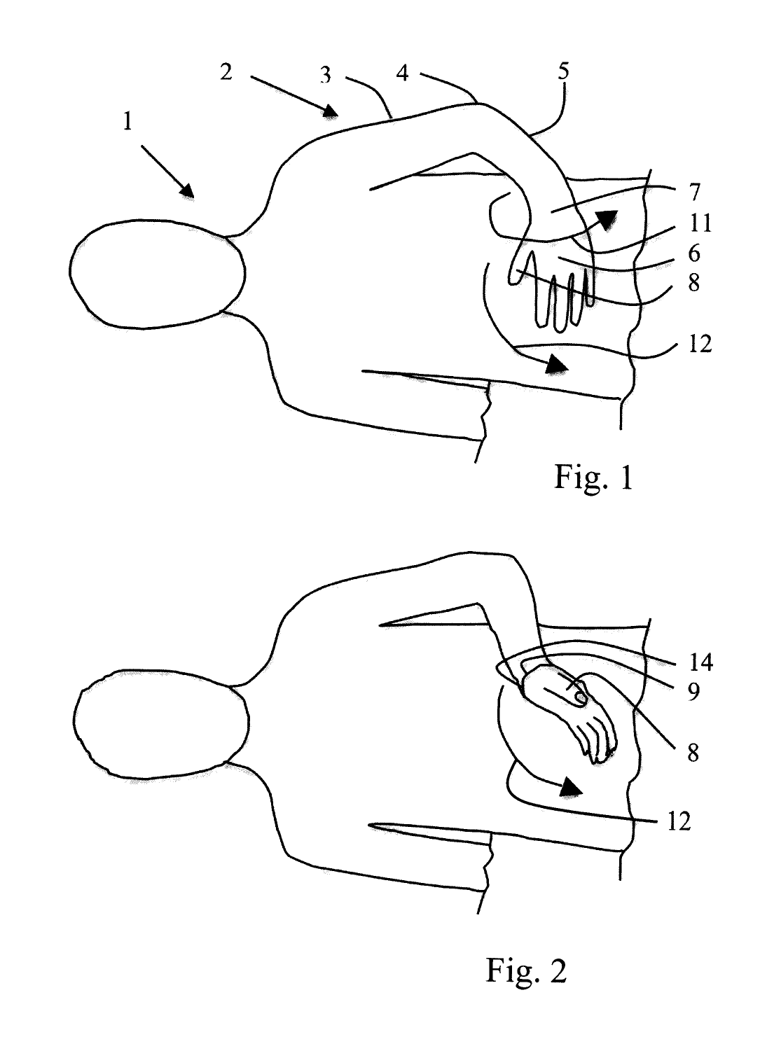

FIG. 1 shows a patient 1 arranged in a supine position with his left arm 2 resting on the abdomen with his hand 6 approximately at the navel. The arm 2 has an upper arm portion 3 and a forearm 5 at each side of the elbow 4. The hand 6 is attached to the forearm 5 via the wrist 7.

The palm of the hand 6 is facing downward towards the abdomen when the patient is in the supine position, as appears from FIG. 1, and the thumb 8 is directed towards the face of the patient. Thus, the palm of the hand and the inner side of the wrist 7 are facing downwards, towards the abdomen and the radial artery is not conveniently available. The palm of the hand is approximately horizontal.

The expressions “inner” and “outer” are intended to mean positions at the normal arm position. Thus, the inner side of the arm is the side of the arm normally facing the abdomen, for example in the position shown in FIG. 1. The outer side of the arm is the portion of the arm facing away from the abdomen. The inner side of the hand, i.e. the palm, is facing the abdomen in the position shown in FIG. 1.

As shown by arrow 11 in FIG. 1, the hand 6 and the wrist 7 may be rotated or twisted about 90 degrees around the symmetry axis of the arm in order to expose the radial artery 9, shown in FIG. 2, at the inner side of the wrist. In this position, the palm surface of the hand forms approximately a right angle with the abdomen and the thumb faces upwards, i.e. the palm of the hand is oriented in a substantially vertical direction when the patient is arranged in the supine position shown in FIGS. 1 and 2. In this twisted position of the arm and the wrist, the radial artery 9 of the left arm is conveniently available for insertion of a catheter in the artery as shown in FIG. 2.

In addition, the wrist should be turned so as to open the angle of the wrist, as shown by arrow 12. The expression “opening” the angle of the wrist is intended to mean that the angle between the forearm 5 and the palm of the hand 6 becomes larger than 180 degrees. In this way the radial artery 9 will be conveniently available.

As an alternative to the radial artery, the surgeon may use an ulnar artery 14 arranged at the position shown in FIG. 2. The ulnar artery is also conveniently available.

It is noted that in FIG. 2 the elbow angle and the wrist angle are arranged in substantially the same plane, but in different directions.

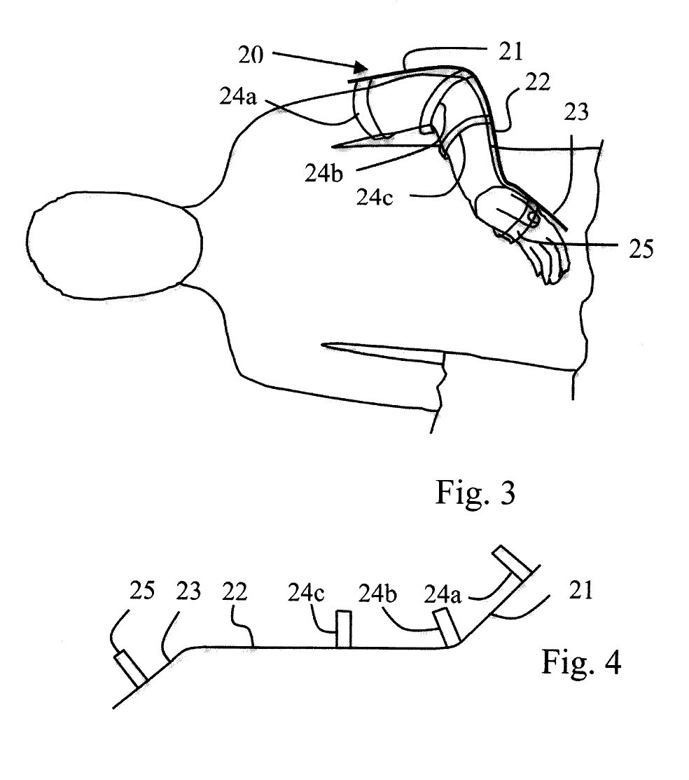

Thus, a fixture with the shape shown in FIG. 3 may be used for arranging the arm in the desired position as indicated above.

The fixture shown in FIG. 3 comprises three interconnected portions, viz. a first portion 21, a second portion 22 and a third portion 23.

The first portion 21 is intended to be arranged at the outside of the upper arm as shown and extending to the elbow.

The second portion 22 is connected to the first portion 21 at a first angle, which may be about 150 degrees. The second portion 22 has a length corresponding to the forearm 4 and supports the forearm. The second portion 22 may have an adjustable length in order to suit patients having different arm lengths.

The third portion 23 is connected to the second portion 22 at a second angle, which is opposite to the first angle and may be of the same size but in the opposite direction. Thus, the second angle is about 210 degrees. The third portion 23 extends along the hand back.

The fixture 20 may be connected to the patient by several straps at suitable positions, as shown in FIG. 3. Thus, first, second and third straps 24a, 24b, 24c may be arranged in a position at the middle of the upper arm 3. A hand strap 25 may be arranged adjacent the hand. The hand strap 25 may be arranged to include the thumb as shown in FIG. 3 or may be arranged below the thumb. The fixture 20 is shown in side view in FIG. 4.

However, the three straps 24a, 24b, 24c will be arranged at the inner side of the upper arm 3 and forearm 5 as shown in FIG. 3. These straps have a tendency to squeeze the artery underlying the straps. The radial artery 9 and also the ulnar artery 14 extend between the skin of the inner portion of the arm and the bone of the arm.

The forces of the straps are distributed over a relatively small surface, which means that the straps penetrate into the underlying skin, tissue and muscles. Especially the second strap 24b may penetrate deeply into the underlying tissue at the inner portion of the elbow and influence upon the arteries there below, but also the first strap 24a and the second strap 24c will provide a strangling action, especially when the fixture is exposed to tension.

Thus, the artery is partially strangled by the straps, which means that a catheter that should be inserted in the artery will have difficulties in passing said strangled areas of the artery. There is also a risk that the artery wall will be scraped or damaged when the catheter is inserted or manipulated.

Thus, there is a need for a fixture, which does not strangle the artery.

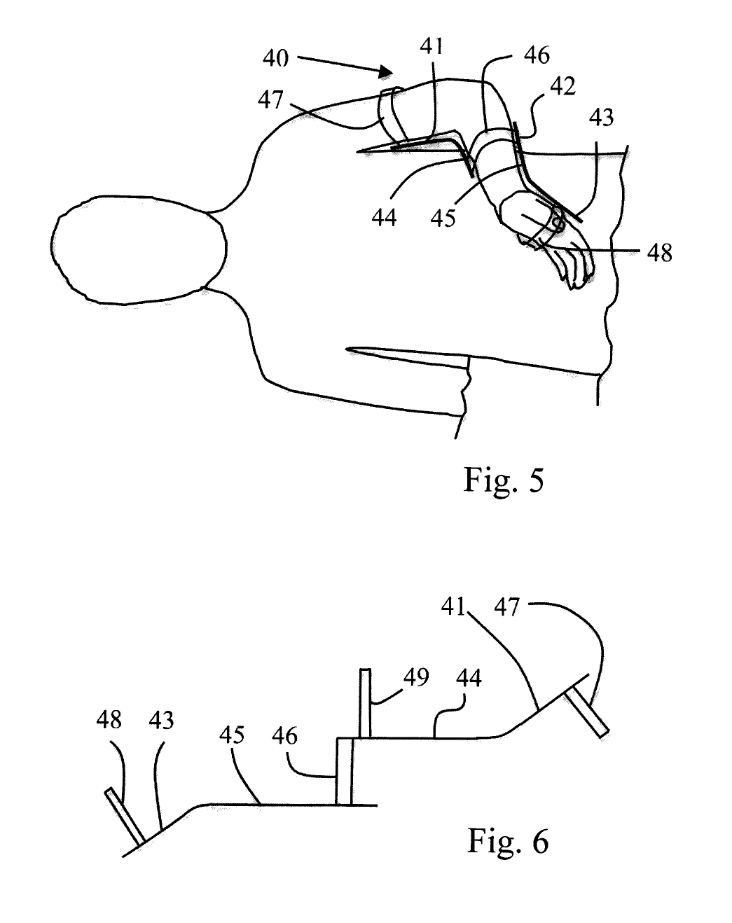

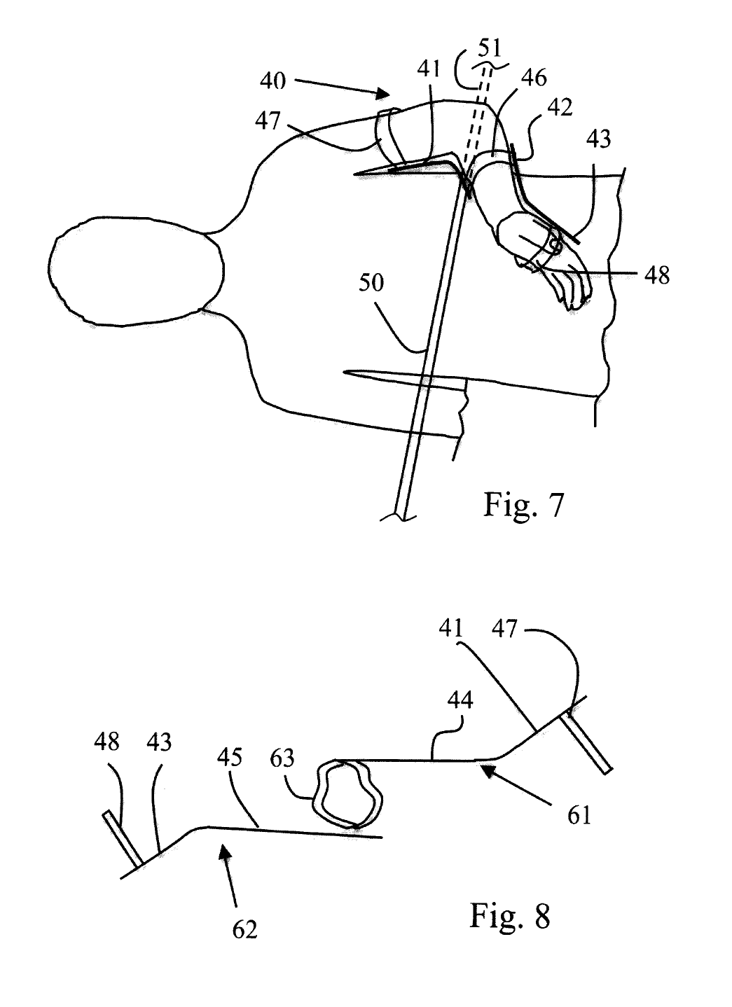

In a first embodiment of the fixture, the above-mentioned problem may be counteracted by providing a fixture, wherein the first portion 21 and a part of the second portion are arranged at the inner side of the elbow, as shown in FIG. 5. In this manner, the forces from the fixture towards the inner surface of the arm will be distributed over the entire surface of the fixture toward the arm and the skin, and the strangling action of the straps will be exerted at the outer side of the arm, where the strangling action does not influence upon the arteries.

The fixture 40 according to the first embodiment comprises three interconnected portions, viz. a first portion 41, a second portion 42 and a third portion 43.

The first portion 41 is intended to be arranged at the inside of the elbow as shown in FIG. 5 and extending along the upper arm portion 3. The first portion 41 is connected to the second portion 42 at a first angle, which may be about 150 degrees. The third portion 43 is connected to the second portion 42 at a second angle, which may be about 210 degrees.

The second portion 42 is divided into two parts, a first part 44 connected to the first portion 41, and a second part 45 connected to the third portion 43. The first part 44 and the second part 45 are interconnected by a half bracelet 46, which encircles half or about 180 degrees of the forearm 4, such as at the outer side of the forearm 4, as shown in FIG. 6. Thus, the half bracelet is called a semi-bracelet below. The semi-bracelet is relatively stiff and is fixedly connected to both the first part 44 and the second part 45. Thus, the fixture 40 forms an integral, relatively rigid, single unit.

By means of the bracelet, the first part 46 and the second part 45 are arranged in parallel but off-set by a distance. The distance substantially corresponds to the diameter of the arm. By this arrangement, the first part 46 may be arranged at the inner side of the arm while the second part 45 is arranged at the outer side of the arm.

However, the attachment of the second part 45 to the bracelet 46 may be adjustable in the longitudinal direction in order to accommodate arms of different lengths.

Alternatively, the first part 44, or both the first and the second part are adjustably connected to the bracelet for adjustment purpose.

The first, second and third portions may have a transversal dimension so that it encircles a portion of the arm, such as about one sixth (60 degrees) of the periphery of the arm. The fixture portion facing the arm may be provided with soft material, so that the forces from the fixture to the arm are evenly distributed. The width of the fixture perpendicular to or transversal to the arm symmetry axis may be about 4 to 12 cm, such as about 6 to 10 cm, for example 8 cm.

The surface of the fixture facing the arm may be provided with a shallow recess extending in the longitudinal direction, so that the arm can rest conveniently in the recess.

The fixture 40 may be connected to the patient by several straps at suitable positions, as shown in FIG. 5. Thus, a first strap 47 may be arranged at a position at the middle of the upper arm 3. A second strap 48 may be arranged adjacent the hand. A third strap (not shown) may be arranged adjacent the semi-bracelet and encircling the entire forearm outside the fixture.

The fixture may be arranged at the patient when the arm is resting along the side of the patient. Alternatively, the arm is brought in the desired position and the fixture is arranged at the arm.

The fixture according to the first embodiment may follow the curvature of the body in a convenient manner without interference with the body.

The fixture according to the first embodiment is shown in a side view in FIG. 6. The third strap 49 is shown slightly beside the semi-bracelet 46, but it may be arranged in any desired position in which it attaches the forearm to the fixture.

It may be desired to move the arm, after being arranged in the fixture, to a position in which the arm and the wrist is more conveniently available. For this purpose, a strap 50 is attached to the first portion 41 and/or the second portion 42, for example at the first part 44 adjacent the semi-bracelet 46. The strap 50 extends over the body to the right side of the patient, wherein the surgeon may pull the strap 50 in order to move the arm towards himself.

The strap 50 may be attached to the bed of the patient in order not to extend outside the bed.

The strap 50 may be arranged to exert a pulling force to the arm in order to arrange the arm in a desired position and counteract a tendency of the arm to fall back to the left side of the body.

Such a pulling force will at the same time relieve the inner surface of the arm from any pressure from the fixture, thereby further preventing any influence upon the underlying artery.

The strap 50 is attached to the fixture in a position as close to the body as possible, in order not to rotate the arm in a non-desired direction, in which the wrist becomes less available.

The strap 50 may in addition extend in the other direction, as shown by broken lines 51, whereupon a nurse arranged at the left side of the patient may interact with the strap, for example to stretch the strap.

The strap 50 may be arranged to pass below the bed to the right side of the bed, so that the surgeon can move the fixture and the arm in both directions. In this arrangement, a nurse at the left side of the patient may interact with the strap portion 51 in order to move the arm towards the surgeon by pulling in the portion extending below the bed.

The strap 50 may be attached to the fixture at any position, which makes it operate as desired. However, if the strap is attached close to the third portion or at the third portion 43, the strap 50 may interact with the catheter to be inserted in the artery of the patient, and, thus, such a position is sometimes non-desired. In addition, the strap may be contaminated with blood.

Although there is presently no requirement for sterilization of the fixture, there may be reasons to keep the fixture as non-contaminated as possible, in order to avoid the introduction of germs or bacteria close to the insertion point of the catheter.

In a second embodiment shown in FIG. 8, the fixture is made up of two separate members, a first member 61 comprising the first portion 41, the first part 44 of the second portion 42 and the semi-bracelet. A second member 62 comprises the second part 45 of the second portion 42 and the third portion 43. The second member is attached to the first member by means of fasteners of any type, such as snap fasteners, a Welcro fastener or a fastener similar to a fastener shown below with reference to FIG. 11.

The division of the fixture in two members 61 and 62 makes it possible to sterilize the second member 62, which is close to the insertion point of the catheter. Thus, the second member 62 may be disposable. This is also an advantage in the case wherein blood has contaminated the second member.

The first member 61 may be reusable and may be made of a material, which may be cleaned in a suitable manner.

It is noted that the entire fixture is made of a material, which is transparent to X-rays, which excludes metals. Thus, the fixture may be made of a plastics material, such as reinforced epoxy plastics material. The second member 62 is made of a material, which may be sterilized.

The fixture may comprise small details of a non-transparent material, such as screws or rivets or metal, but such details should be arranged in areas so that they do not interfere with the blood vessels to be examined or used.

In the first and the second embodiment, the semi-bracelet does not need to be rigid. The semi-bracelet may be made of a flexible material that has a certain spring action so that the bracelet can be arranged at forearms of different sizes.

In an alternative design shown in FIG. 8, the semi-bracelet 46 is completely replaced by a strap 63, which encircles the forearm approximately at the middle thereof. The strap 63 is flexible but is non-elastic. The strap 63 is attached to the forearm sufficiently rigidly for keeping the forearm in the desired rotational position, but does not strangle the underlying artery, because the inner side of the part 44 has a sufficiently large surface to spread the forces.

The second member 62 is attached to the strap 49 by quick release fasteners. The second member 62 may be attached to the strap 63 at a suitable longitudinal position so that the combined first part 44 and second part 45 corresponds to the length of the forearm.

In one arrangement, the second part 45 is only inserted between the strap 63 and the forearm after the upper portion 44 has been arranged in place.

In certain applications, wherein the requirement on the fixture is less stringent, it may be sufficient to use only the first member 61. In this case, the forearm is fixed in the desired rotational position by the strap 63, which is firmly arranged around the forearm.

The strap 63 may be provided with friction material at the surface facing the arm, in order to hold the forearm in the desired rotational position. The friction material may be rubber or an elastomer. Any of the straps may be provided with such friction material.

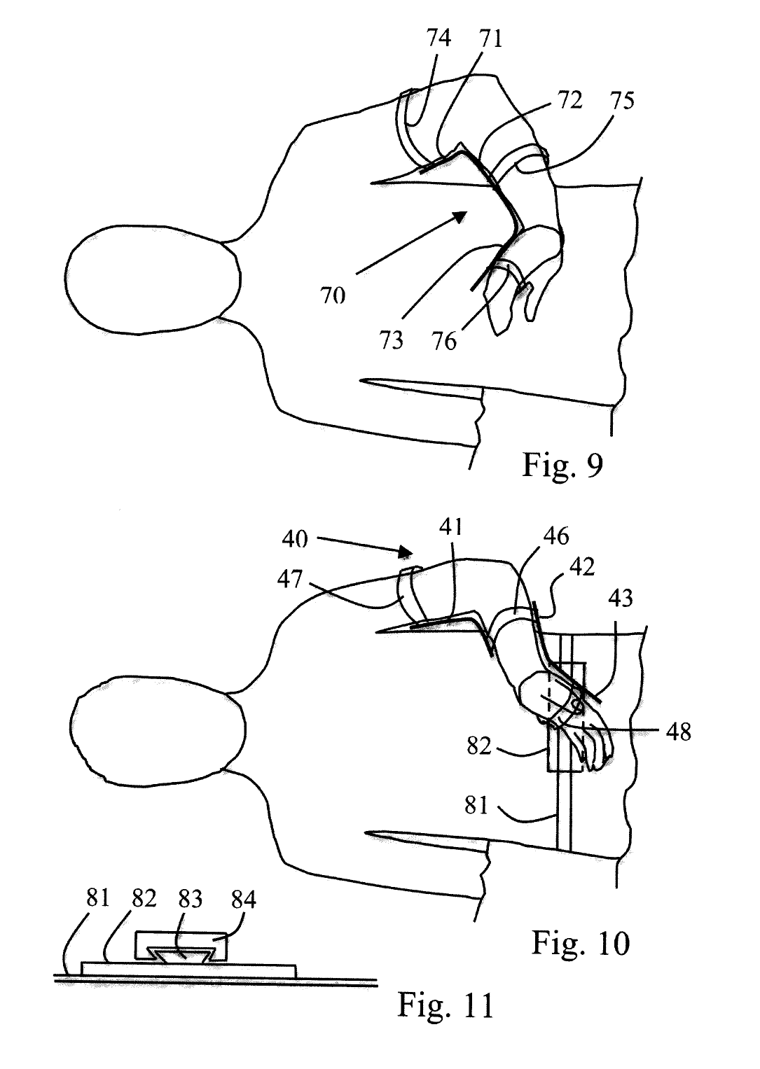

A third embodiment of the fixture is shown in FIG. 9. The fixture 70 is adapted to arrange the arm in a position, in which the ulnar artery 14 may be conveniently available, for example if it is more superficial. Alternatively, the radial artery is also available in this position. In the third embodiment, the hand is rotated in a direction opposite to that shown by arrow 11 in FIG. 1. Thus, the palm of the hand is still vertical and substantially perpendicular to the abdomen, but the thumb faces down towards the abdomen.

The fixture 70 is arranged along the inner side of the upper arm and elbow, and further along the inner side of the forearm and finally along the outer side of the twisted hand, i.e. the hand back.

The arrangement is similar to the second embodiment, but the second angle is in the same direction as the first angle, i.e. the second angle is about 150 degrees.

The fixture according to the third embodiment comprises a first portion 71 extending along the inside of the upper arm, a second portion 72 extending along the rotated forearm, and a third portion 73 extending along the outer side of the hand. Several straps 74, 75 and 76 connect the fixture to the arm.

The straps may be made of a flexible material and having a length corresponding to the diameter of the respective arm portion. The end of the strap may be provided with a fastening means, such as a Velcro strap. The fixture may be provided with a Velcro fastener at a suitable position in order to interact with the Velcro strap.

Alternatively, two straps may be arranged extending in opposite directions and ending with mating Velcro fasteners.

There may be arranged additional semi-bracelets in order so support the fixture in relation to the arm. The supporting semi-bracelet may extend over 180 degrees or over still smaller angle such as 120 degrees or 90 degrees. Thus, a supporting bracelet may be arranged at the middle of the second part 44 and encircling half the arm at the outer side thereof.

The fixture may be made of glass-fiber reinforced plastics material, such as an epoxy plastics material. Other suitable materials may be PVC and polyurethane. The material should be of the type, which is permitted to be used in medical applications. The material should be transparent to X-ray radiation.

The fixture may be covered by textile at the surfaces facing the arm or the body. The straps may also be covered by a soft material or a material having friction towards the arm or the skin, such as rubber or an elastomer.

A belt may be arranged around the waist of the patient as shown in FIG. 10. FIG. 10 shows the patient having a belt 81 around his waist. The belt is arranged loosely at the patient, so that it can be moved in the circumferential direction. A plate 82 is attached to the belt in a position corresponding to the navel of the patient. The belt comprises a fastening member (not shown) at a position close to the navel. The fixture comprises a mating fastening member (not shown), so that the fixture may be attached to the fastening member of the belt. The belt 81 is available at the right side and at the left side of the patient. Thus, the exact circumferential position of the fixture may be adjusted during use by pulling the belt 81. Thus, the fastening means may be moved towards the surgeon at the right side of the patient during insertion of the catheter by pulling the belt at the right side of the patient. When the catheter is in place, the fastening means may be moved away from the surgeon by pulling the belt at the left side of the patient.

The fastening member of the belt plate 82 may be a protrusion 83 attached to the plate 82 and having undercut grooves at two sides thereof. The fastening member at the fixture may be a protrusion 84 having a mating shape for engagement with the protrusion 83 of the belt, as shown in FIG. 11.

Another alternative fastening member may be a hitch and hook. Still another alternative fastening member may be a Velcro fastener.

By making the fixture immovable by said belt adjacent the navel, the fixture will be out of the way in an emergency case, when for example a defibrillation is required.

In certain applications, it may be sufficient to use the second member 62, as shown in FIG. 8 attached to the belt plate 82, whereby the first member 61 can be dispensed with.

Further alternative combinations of the different features shown in the different embodiments are possible within the scope of the invention.

The first angle may be from about 170 degrees to about 110 degrees. The second angle may be from about 190 degrees to about 250 degrees. In the third embodiment, the second angle may be from about 170 degrees to 110 degrees. The first and second angles may be adjustable.

The different embodiments have been described in an application wherein a catheter should be introduced into the radial or ulner artery of the left arm. Such catheter may be used in heart investigations and intervention.

There are other medical methods when it may be desired to immobilize the left arm in the position shown, for example in emergency situations when fast and convenient access to the arm artery or vein is desired.

Another medical method may be the introduction of cold saline in the arm artery for cooling of the body as soon as possible after a brain injury, such as brain hemorrhage or stroke. About 500 to 1000 ml cold saline is introduced in the body as soon as possible to induce hypothermia.

A further medical method in which the fixture may be used is the localization and dissolution of clots or obstructions in other positions than the heart.

In the claims, the term “comprises/comprising” does not exclude the presence of other elements or steps. Furthermore, although individually listed, a plurality of means, elements or method steps may be implemented by e.g. a single unit. Additionally, although individual features may be included in different claims or embodiments, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

Although the present invention has been described above with reference to specific embodiment and experiments, it is not intended to be limited to the specific form set forth herein. Rather, the invention is limited only by the accompanying claims and, other embodiments than those specified above are equally possible within the scope of these appended claims.

1. A fixture for being attached to an arm of a patient, comprising a first portion, a second portion and a third portion,
the first portion being intended to be arranged adjacent an elbow at the inner side of the elbow and extending from the elbow up along an upper arm portion and being arranged integral with the second portion at a first angle;
the second portion having a length which is smaller than or equal to a forearm and supporting the forearm;
the third portion being integral with or connected to the second portion at a second angle.

2. The fixture according to claim 1, wherein said second angle is in a direction opposite the first angle, and is about 190 degrees to 250 degrees and wherein the first and second angles are in substantially the same plane and wherein the fixture substantially forms a Z-shape.

3. The fixture according to claim 1, wherein said second angle is in the same direction as the first angle, and is about 110 degrees to 170 degrees and wherein the first and second angles are in substantially the same plane and wherein the fixture forms a ground U-shape.

4. The fixture according to claim 1, wherein said fixture is attached to the arm of the patient by several flexible straps.

5. The fixture according to claim 1, wherein the length of the second portion is adjustable.

6. The fixture according to claim 1, wherein the second portion is divided in two parts interconnected by a semi-bracelet, whereby a first part of the second portion is arranged integral with said first portion by said first angle and a second part of the second portion is integral with or connected to the third portion at said second angle.

7. The fixture according to claim 6, wherein the semi-bracelet extends over about 180 degrees around a symmetry axis of the arm, whereby the first part is arranged at the inner side of the arm and the second part is arranged at the outer side of the arm.

8. The fixture according to claim 1, wherein at least a portion of the fixture is substantially transparent to X-ray radiation.

9. The fixture according to claim 1, further comprising a fastening means for fastening the fixture in relation to the body of the patient, such as a fastening means adjacent the third portion intended for attachment to a belt.

10. The fixture according to claim 1, wherein at least a member of the fixture is sterilized.

11. A method for immobilizing an arm of a patient at a position adjacent a navel of the patient and with a wrist accessible by having the wrist arranged in a twisted position with the thumb facing upwards or downwards when the patient is in a supine position, in order to facilitate insertion of a catheter in an artery of the arm for heart investigation or intervention.

12. The method according to claim 11, in which the wrist is angled so that the wrist is opened up.

13. The method according to claim 11, wherein the arm is immobilized by attaching the arm to a fixture, comprising a first portion, a second portion and a third portion, wherein
the first portion is arranged adjacent an elbow at the inner side of the elbow and extending from the elbow up along an upper arm portion and being arranged integral with the second portion at a first angle;
the second portion supports the forearm and have a length which is smaller than or equal to the forearm;
the third portion being integral with or connected to the second portion at a second angle and supports the wrist and hand at the outer side thereof.
